(19) United States
(12) Patent Application Publication
(10) Pub. No.: US 2026/0207134 A1
(43) Pub. Date: Jul. 23, 2026

(54) INTELLIGENT JOINT MONITORING DEVICE AND METHOD INTEGRATING IN-VITRO MULTI-NODE WEARABLE MODULE AND IN-VIVO IMPLANTABLE INTELLIGENT JOINT PROSTHESIS

(71) Applicant: Beihang University, Beijing (CN)

(72) Inventors: Xing CHEN, Beijing (CN); Zhixiong ZHAO, Beijing (CN)

(21) Appl. No.: 19/449,714

(22) Filed: Jan. 15, 2026

(30) Foreign Application Priority Data
Jan. 20, 2025 (CN) 202510084945.4

(51) Int. Cl. A61B 5/00; A61B 5/11; A61B 90/00; A61F 2/32; A61F 2/38; A61F 2/48

(52) U.S. Cl. CPC A61B 5/4851; A61B 5/1121; A61B 5/1126; A61B 5/4528; A61B 5/4848; A61B 5/686; A61B 5/7203; A61B 5/7271; A61B 90/06; A61F 2/32; A61F 2/38; A61F 2/482; A61B 2560/0219; A61B 2560/0462; A61B 2562/0219; A61B 2562/0271

(57) ABSTRACT

This application provides an intelligent joint monitoring device and method integrating an in-vitro multi-node wearable module and an in-vivo implantable intelligent joint prosthesis, relating to the field of joint monitoring. The device includes: an in-vitro multi-node wearable module and an in-vivo implantable intelligent joint prosthesis. The in-vitro multi-node wearable module and the in-vivo implantable intelligent joint prosthesis cooperatively analyze a rehabilitation status of a patient with an implanted artificial joint. The in-vitro multi-node wearable module includes a first main control chip, a first inertial measurement unit (IMU) chip and a Bluetooth chip. The in-vivo implantable intelligent joint prosthesis includes a joint prosthesis body, and a second main control chip, a second IMU chip and a near field communication (NFC) tag chip that are arranged in the joint prosthesis body.

### CROSS REFERENCE TO RELATED APPLICATION

This patent application claims the benefit and priority of Chinese Patent Application 202510084945.4, entitled “INTELLIGENT JOINT MONITORING DEVICE AND METHOD INTEGRATING IN-VITRO MULTI-NODE WEARABLE MODULE AND IN-VIVO IMPLANTABLE INTELLIGENT JOINT PROSTHESIS” and filed with the China National Intellectual Property Administration on Jan. 20, 2025, the disclosure of which is incorporated by reference herein in its entirety as part of the present application.

### TECHNICAL FIELD

This application relates to the field of joint monitoring, and in particular to an intelligent joint monitoring device and method integrating an in-vitro multi-node wearable module and an in-vivo implantable intelligent joint prosthesis.

### BACKGROUND

Joint prostheses primarily focus on wear resistance and biocompatibility of materials, lacking real-time monitoring capabilities for joint movements and the biological environment. Postoperative monitoring methods, such as periodic clinical examinations and imaging assessments, cannot provide real-time data or monitor actual performance of the prosthesis during daily activities.

In some cases, it is impossible to provide real-time data of the prosthesis during use, so it is impossible to adjust the rehabilitation plan or predict potential problems in time. In some cases, monitoring methods require the patient to have examinations periodically in hospitals or clinics, which may affect the compliance and quality of life of the patient.

In addition, a patient who has undergone joint replacement unconsciously uses the healthy leg for compensation in daily activities due to the presence of the prosthesis, leading to the altered walking posture, sitting posture and movement posture. Therefore, monitoring of kinematics relying solely on sensors integrated with the wearable device or prosthesis may lead to the loss of key information of other parts.

### SUMMARY

An objective of this application is to provide an intelligent joint monitoring device and method integrating an in-vitro multi-node wearable module and an in-vivo implantable intelligent joint prosthesis, which can realize real-time monitoring of a joint and other body parts.

To achieve the above objective, this application provides the following technical solutions.

In a first aspect, this application provides an intelligent joint monitoring device integrating an in-vitro multi-node wearable module and an in-vivo implantable intelligent joint prosthesis, including an in-vitro multi-node wearable module and an in-vivo implantable intelligent joint prosthesis. The in-vitro multi-node wearable module is configured to acquire movement data of a plurality of different in-vitro parts to form overall body posture and movement status data. The in-vivo implantable intelligent joint prosthesis is configured to acquire interarticular relative local movement status data. The in-vitro multi-node wearable module and the in-vivo implantable intelligent joint prosthesis cooperatively analyze a rehabilitation status of a patient with an implanted artificial joint. The in-vitro multi-node wearable module includes a first main control chip, a first inertial measurement unit (IMU) chip and a Bluetooth chip. The first IMU chip is configured to acquire in-vitro data. The first main control chip is connected to the first IMU chip. The first main control chip is configured to preprocess the in-vitro data. The Bluetooth chip is connected to the first main control chip. The Bluetooth chip is configured to wirelessly broadcast the preprocessed in-vitro data to a mobile terminal. The in-vivo implantable intelligent joint prosthesis includes a joint prosthesis body, and a second main control chip, a second IMU chip and a near field communication (NFC) tag chip that are arranged in the joint prosthesis body. The second IMU chip is configured to acquire joint movement data in real time. The second main control chip is connected to the second IMU chip. The second main control chip is configured to preprocess and encrypt the joint movement data. The NFC tag chip is connected to the second main control chip. The NFC tag chip is configured to store the encrypted joint movement data.

In a second aspect, this application provides an intelligent joint monitoring method integrating an in-vitro multi-node wearable module and an in-vivo implantable intelligent joint prosthesis, including: acquiring, by an in-vitro multi-node wearable module, in-vitro data; preprocessing the in-vitro data; wirelessly broadcasting preprocessed in-vitro data to a mobile terminal; acquiring, by an in-vivo implantable intelligent joint prosthesis, joint movement data, where the joint movement data includes knee joint movement data and hip joint movement data; preprocessing the joint movement data, where the preprocessing includes filtering and denoising; encrypting preprocessed joint movement data; and transmitting the encrypted joint movement data to the mobile terminal.

According to specific embodiments provided in this application, this application has the following technical effects:

This application provides the intelligent joint monitoring device and method integrating an in-vitro multi-node wearable module and an in-vivo implantable intelligent joint prosthesis. The in-vitro data are collected in real time by the first IMU chip in the in-vitro multi-node wearable module, and the joint movement data are collected in real time by the second IMU chip in the in-vivo implantable intelligent joint prosthesis, thereby realizing real-time monitoring of the knee/hip joint and other body parts. Real-time monitoring of data enables the clinician to adjust the rehabilitation plan in time and provide the patient with an individualized treatment schedule, thereby accelerating the rehabilitation process. In addition, the in-vitro multi-node wearable module and the in-vivo implantable intelligent joint prosthesis cooperate to collect multidimensional sensing information, which enables comprehensive monitoring of the human body and facilitates multidimensional analysis and targeted rehabilitation for the patient.

### BRIEF DESCRIPTION OF THE DRAWINGS

To describe the technical solutions in the embodiments of this application or in the prior art more clearly, the following briefly describes the accompanying drawings required for the embodiments. Apparently, the accompanying drawings in the following description show merely some embodiments of this application, and a person of ordinary skill in the art may still derive other accompanying drawings from these accompanying drawings without creative efforts.

FIG. 1 is a schematic diagram of function modules of an intelligent joint monitoring device integrating an in-vitro multi-node wearable module and an in-vivo implantable intelligent joint prosthesis according to an embodiment of this application;

FIG. 2 is a schematic view showing positions of the in-vitro multi-node wearable module and the in-vivo implantable intelligent joint prosthesis according to an embodiment of this application.

FIG. 3 is a schematic structural view of a total knee joint prosthesis according to an embodiment of this application.

FIG. 4 is a schematic structural view of a hip joint prosthesis according to an embodiment of this application.

FIG. 5 is a schematic view showing cooperation of a wearable module outside a total knee joint prosthesis and an in-vivo implantable intelligent total knee joint prosthesis according to an embodiment of this application.

FIG. 6 is a flowchart showing an intelligent joint monitoring method integrating an in-vitro multi-node wearable module and an in-vivo implantable intelligent joint prosthesis according to an embodiment of this application.

FIG. 7 is a schematic view of a 6-degree-of-freedom (DOF) linkage and Denavit-Hartenberg (D-H) coordinates according to an embodiment of this application.

### DETAILED DESCRIPTION OF THE EMBODIMENTS

The technical solutions in the embodiments of this application are clearly and completely described below with reference to the drawings in the embodiments of this application. Apparently, the described embodiments are only some rather than all of the embodiments of this application. All other embodiments obtained by a person of ordinary skill in the art based on the embodiments of this application without creative efforts shall fall within the protection scope of this application.

To make the above objectives, features, and advantages of this application more obvious and easy to understand, this application will be further described in detail with reference to the accompanying drawings and specific implementations.

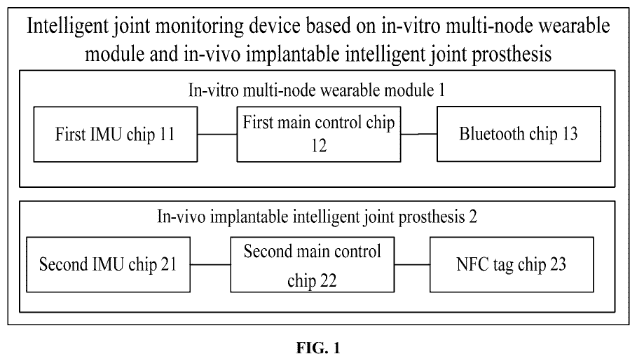

In Embodiment 1, as shown in FIG. 1, an intelligent joint monitoring device integrating an in-vitro multi-node wearable module and an in-vivo implantable intelligent joint prosthesis is provided, including an in-vitro multi-node wearable module 1 and an in-vivo implantable intelligent joint prosthesis 2. The in-vitro multi-node wearable module 1 is configured to acquire movement data of a plurality of different in-vitro parts to form overall body posture and movement status data. The in-vivo implantable intelligent joint prosthesis 2 is configured to acquire interarticular relative local movement status data. The in-vitro multi-node wearable module 1 and the in-vivo implantable intelligent joint prosthesis 2 cooperatively analyze a rehabilitation status of a patient with an implanted artificial joint. The in-vitro multi-node wearable module 1 and the in-vivo implantable intelligent joint prosthesis 2 form multi-point linkage to collect and analyze data.

The in-vitro multi-node wearable module 1 includes a first main control chip 12, a first inertial measurement unit (IMU) chip 11 and a Bluetooth chip 13. The first IMU chip 11 acquires in-vitro data (in-vitro data such as triaxial angular velocity and triaxial acceleration). The first main control chip 12 is connected to the first IMU chip 11. The first main control chip 12 is configured to preprocess the in-vitro data. The Bluetooth chip 13 is connected to the first main control chip 12. The Bluetooth chip 13 is configured to wirelessly broadcast the preprocessed in-vitro data to a mobile terminal.

The in-vitro multi-node wearable module 1 further includes a lithium battery. The lithium battery is configured to supply power to the first main control chip 12, the first IMU chip 11 and the Bluetooth chip 13.

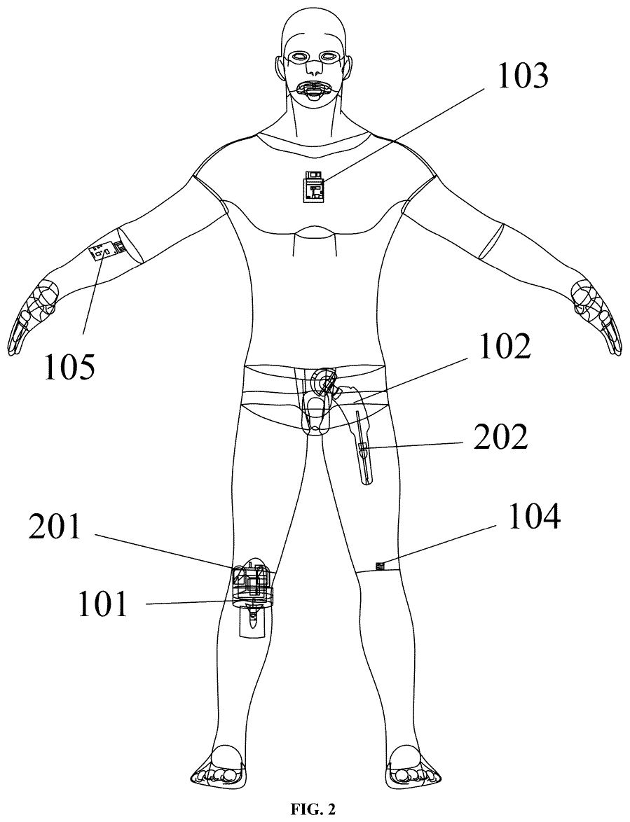

As shown in FIG. 2, the in-vitro multi-node wearable module 1 includes a wearable module outside the joint prosthesis and a wearable module for other body parts. The wearable module outside the joint prosthesis includes a wearable module 101 outside a total knee joint prosthesis and a wearable module 102 outside a hip joint prosthesis. The wearable module for other body parts is disposed in different positions as required, mainly on the torso, healthy leg and arm. The wearable module for other body parts includes a torso wearable module 103, a leg wearable module 104 and an arm wearable module 105. The wearable modules in the in-vitro multi-node wearable module 1 communicate through the Bluetooth chip 13.

The wearable module outside the joint prosthesis further includes an NFC read/write chip, a power amplifier, a power supply inductive coil Q2 and a read inductive coil Q1. The lithium battery supplies power to the in-vivo implantable intelligent joint prosthesis 2 by electromagnetic coupling and resonance through the power amplifier and the power supply inductive coil Q2. The read inductive coil Q1 reads information stored in the NFC read/write chip by 13.56 MHz resonance coupling, including information acquired by a triaxial accelerometer, a gyroscope, a magnetometer, a temperature sensor and a pH sensor.

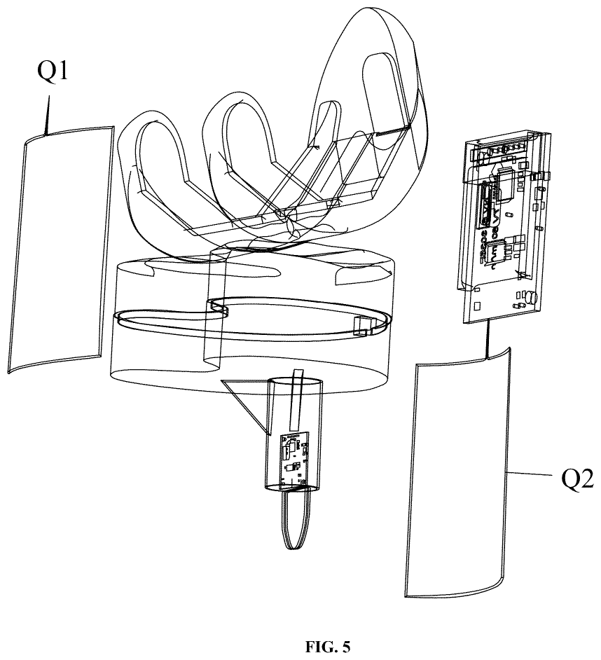

The inductive coils (the power supply inductive coil Q2 and the read inductive coil Q1) of the wearable module outside the joint prosthesis are disposed on the leg on two outer sides of a tibial component prosthesis or a femoral component prosthesis, and the two sides directly face the inductive coils arranged in the joint prosthesis body of the in-vivo implantable intelligent joint prosthesis 2. FIG. 5 is a schematic view showing cooperation of the wearable module 101 outside a total knee joint prosthesis and an in-vivo implantable intelligent total knee joint prosthesis 201. The cooperation of the wearable module 102 outside a hip joint prosthesis and the in-vivo implantable intelligent hip joint prosthesis 202 is similar.

The in-vivo implantable intelligent joint prosthesis 2 includes a joint prosthesis body, and a second main control chip 22, a second IMU chip 21 and an NFC tag chip 23 that are arranged in the joint prosthesis body. The second IMU chip 21 is configured to acquire joint movement data in real time. The second main control chip 22 is connected to the second IMU chip 21. The second main control chip 22 is configured to preprocess and encrypt the joint movement data. The NFC tag chip 23 is connected to the second main control chip 22. The NFC tag chip 23 is configured to store the encrypted joint movement data. An inductive coil is arranged in the joint prosthesis body and configured to receive electrical energy transferred by the power supply inductive coil Q2.

As shown in FIG. 2, the in-vivo implantable intelligent joint prosthesis 2 includes an in-vivo implantable intelligent total knee joint prosthesis 201 and an in-vivo implantable intelligent hip joint prosthesis 202. The joint prosthesis body includes a total knee joint prosthesis body and a hip joint prosthesis body.

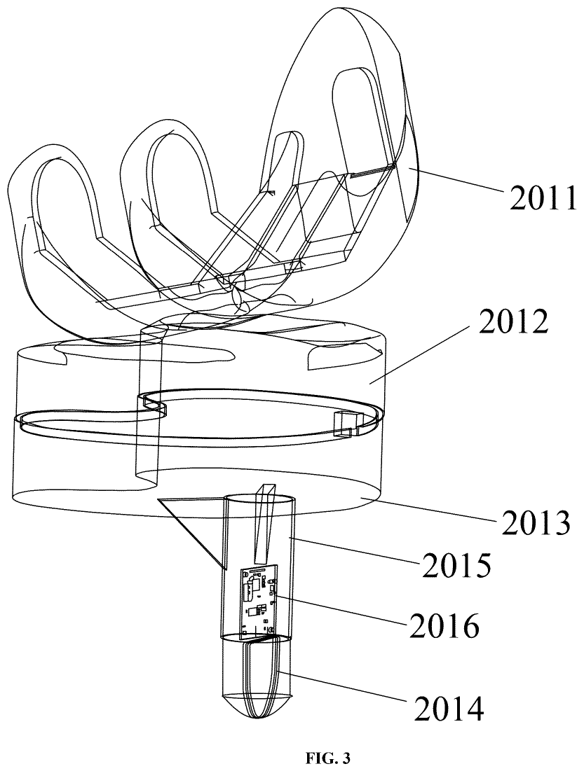

As shown in FIG. 3, the total knee joint prosthesis body includes a femoral component prosthesis 2011, a tibial liner prosthesis 2012 and a tibial component prosthesis 2013. A temperature sensor, an air pressure sensor, a pH sensor, an impedance sensor and a blood pressure and blood oxygen chip and necessary electronic components are further arranged in the total knee joint prosthesis body.

The second IMU chip 21, the temperature sensor, the pressure sensor, the pH sensor and the impedance sensor in the implantable intelligent total knee joint prosthesis 201 are configured to monitor multidimensional sensing information in vivo and transmit the acquired data to the second main control chip 22 based on communication protocols such as SPI and I2C. The second main control chip 22 then transmits the data to an EEPROM of the NFC tag chip 23 based on I2C. After the storage space of the NFC tag chip 23 is full, the information in the memory is overwritten cyclically. The implantable intelligent total knee joint prosthesis 201 wirelessly transmits electrical energy to the inductive coil 2014 of the implantable intelligent total knee joint prosthesis 201 through the power supply inductive coil Q2 in the wearable module 101 outside a total knee joint prosthesis. In-vivo wireless alternating current is rectified and filtered by the NFC tag chip 23 to output direct current, which powers the modules in the implantable intelligent total knee joint prosthesis 201.

All the sensors of the implantable intelligent total knee joint prosthesis 201 are integrated on a circuit board 2016. The circuit board 2016 is arranged in a cavity of a tibial stem prosthesis 2015. The circuit board 2016 is disposed at an upper part of the tibial stem prosthesis 2015 to minimize interference with other parts of the knee joint. The inductive coil 2014 is located at a lower portion of the tibial stem prosthesis 2015 and is encapsulated together with the tibial stem prosthesis in a transparent plastic component. The antenna is designed to follow the contour of the tibial stem prosthesis 2015 to maximize the energy reception area.

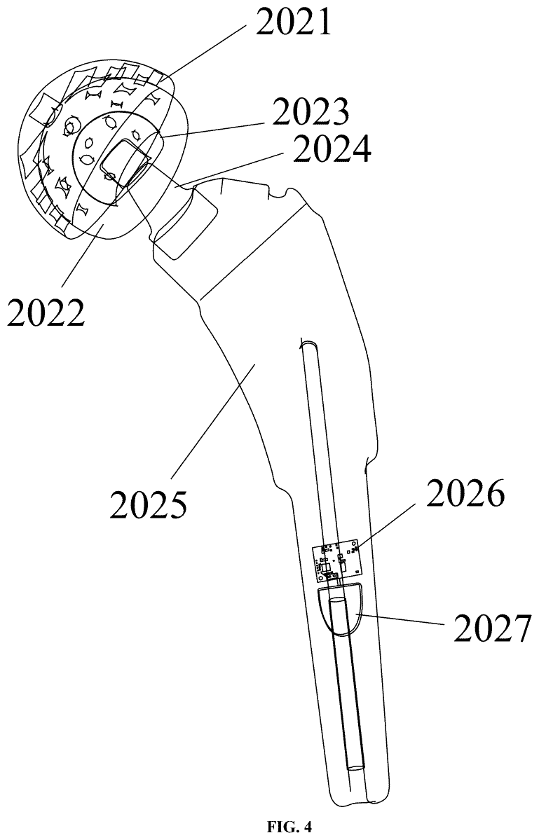

As shown in FIG. 4, the hip joint prosthesis body includes an acetabular prosthesis 2021, acetabular liner prosthesis 2022, a femoral head prosthesis 2023, a femoral neck prosthesis 2024 and a femoral stem prosthesis 2025.

The second IMU chip 21 in the implantable intelligent hip joint prosthesis 202 is configured to monitor motion parameters of the femoral stem prosthesis 2025. Acquired data are subjected to floating point conversion based on IEEE 754 standard, such that the floating points are converted into hexadecimal data. The hexadecimal data are transmitted to the second main control chip 22 based on I2C. The second main control chip 22 stores the data according to the different axes, and then transmits the data into the storage space of the EEPROM of the NFC tag chip 23 based on I2C.

All the sensors and necessary electronic components of the implantable intelligent hip joint prosthesis 202 are integrated on a circuit board 2026. The circuit board 2026 is arranged in a cavity of the femoral stem prosthesis 2025. An inductive coil 2027 of the implantable intelligent hip joint prosthesis 202 is located at a lower end of the femoral stem prosthesis 2025. The antenna is designed to follow the contour of the femoral stem prosthesis 2025 to maximize the energy reception area.

The intelligent joint monitoring device integrating an in-vitro multi-node wearable module and an in-vivo implantable intelligent joint prosthesis provided in this application integrates advanced sensors, wireless communication technology and data processing algorithms to realize real-time monitoring of the hip/knee joint and other body parts and perform multidimensional cross-comparisons. The wearable module and the implantable prosthesis can provide key information regarding stability and functional status of the prosthesis without increasing the burden of the patient, thereby improving the postoperative rehabilitation process and ultimately improving the quality of life of the patient.

Through the above design, real-time data on the hip/knee joint of the patient can be provided to the clinician, so that the treatment plan and the rehabilitation process can be optimized. Moreover, the use of the NFC technology facilitates easier monitoring and maintenance of the prosthesis, thereby improving the quality of life of the patient.

The intelligent joint monitoring device integrating an in-vitro multi-node wearable module and an in-vivo implantable intelligent joint prosthesis provided in this application provides the following advantages:
(1) The device can acquire multidimensional sensing information.
(2) By combining the wearable module and the implantable prosthesis, the device can realize efficient power supply and high-fidelity data storage and transmission.
(3) The device can not only monitor multidimensional in-vivo information, but also realize multidimensional comparison between sensing information of different body parts in vitro and sensing information in vivo, thereby providing the clinician with more precise and convenient access to the information of the patient.
(4) The clinician can conduct comprehensive analyses based on multidimensional sensing information to formulate an individualized and rational treatment plan.
(5) Real-time monitoring: By integrating the IMU, temperature, pH and impedance sensors, the prosthesis can monitor the movement status, internal temperature, pH and tissue impedance of the knee joint, thereby providing the clinician with an innovative postoperative tracking and evaluation tool.
(6) The power supply inductive coil Q2 has high efficiency and power. The shape of the power supply inductive coil Q2 is designed based on the contours of the tibial component and the femoral component and the calculations of the maximum electromagnetic intensity.
(7) Optimization of rehabilitation process: Real-time monitoring of data enables the clinician to adjust the rehabilitation plan in time and provide the patient with an individualized treatment schedule, thereby accelerating the rehabilitation process.
(8) Biocompatibility: All the sensors and electronic components are made of biocompatible materials, thereby reducing potential biological rejection reactions after prosthesis implantation and improving long-term stability of the prosthesis. The adhesive material is epoxy resin, which is non-cytotoxic.

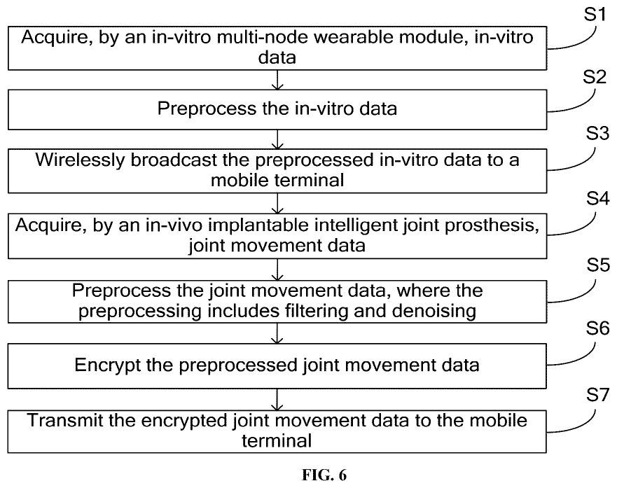

In Embodiment 2, based on the same inventive concept, an embodiment of this application further provides a method applied to the intelligent joint monitoring device integrating an in-vitro multi-node wearable module and an in-vivo implantable intelligent joint prosthesis. As shown in FIG. 6. The intelligent joint monitoring method integrating an in-vitro multi-node wearable module and an in-vivo implantable intelligent joint prosthesis includes:
S1: Acquire, by an in-vitro multi-node wearable module, in-vitro data.
S2: Preprocess the in-vitro data.
S3: Wirelessly broadcast preprocessed in-vitro data to a mobile terminal.
S4: Acquire, by an in-vivo implantable intelligent joint prosthesis, joint movement data, where the joint movement data includes knee joint movement data and hip joint movement data.
S5: Preprocess the joint movement data, where the preprocessing includes filtering and denoising.
S6: Encrypt preprocessed joint movement data.
S7: Transmit the encrypted joint movement data to the mobile terminal.

After step S7, the method further includes:
decrypting the encrypted joint movement data; and monitoring a rehabilitation status of a patient in real time according to the decrypted joint movement data and the in-vitro data, and adjusting a rehabilitation plan of the patient. The rehabilitation status includes an upper limb rehabilitation status and a lower limb rehabilitation status.

In an exemplary embodiment, a process of monitoring the lower limb rehabilitation status includes:
1) establishing a coordinate system based on a Denavit-Hartenberg (D-H) method, building a 6-degree-of-freedom (DOF) linkage, and using the 6-DOF linkage as a lower limb kinematic model, where the 6-DOF linkage has three rotational degrees of freedom, and the linkage is used for characterizing flexion and extension movements of a hip joint and flexion and hyperextension movements of a knee joint;
2) calculating, based on preprocessed in-vitro data of lower limbs, hip and knee joint angles of unaffected and affected legs according to the lower limb kinematic model;
3) training, with gait recognition as an objective, a random forest machine learning algorithm by using the hip and knee joint angles of the unaffected and affected legs and a triaxial acceleration and a triaxial angular velocity in the joint movement data as inputs, and monitoring results of the lower limb rehabilitation status as outputs, where types of the joint movement data include posture angle, the triaxial acceleration, the triaxial angular velocity, temperature, pH and impedance; and
4) obtaining the hip and knee joint angles of the monitored lower limbs and the triaxial acceleration and the triaxial angular velocity in the joint movement data, and recognizing the lower limb rehabilitation status by using the trained random forest machine learning algorithm, to obtain the monitoring results of the monitored lower limb rehabilitation status.

In an exemplary embodiment, this embodiment can provide early warnings of gait abnormalities through the center of mass, perimeter, area and range of motion in a joint angle cycle diagram.

During actual application, the kinematic data used in monitoring of the lower limb rehabilitation status reflects the health status and movement patterns of local joints and bones. The in-vitro wearable module monitors kinematic data of other body parts, including motions of the healthy lower limb and the upper limb. This multi-point acquisition of local data enables comparisons and analysis of movement statuses of different body parts.

Movements of the lower limb primarily involve the combination of movements of the hip and knee joints. Walking mainly involves flexion/extension of the hip joint and flexion/hyperextension of the knee joint. Based on the main movement patterns of the joints during walking, the human lower limb kinematic model is established. Since the main movement of each joint involves rotation around its axis in the sagittal plane, the lower limb can be simplified to a 6-DOF linkage, having three rotational degrees of freedom on each side. The coordinate system is established based on the D-H method, and angular transformations are achieved based on the rotational transformation relationships between the joints.

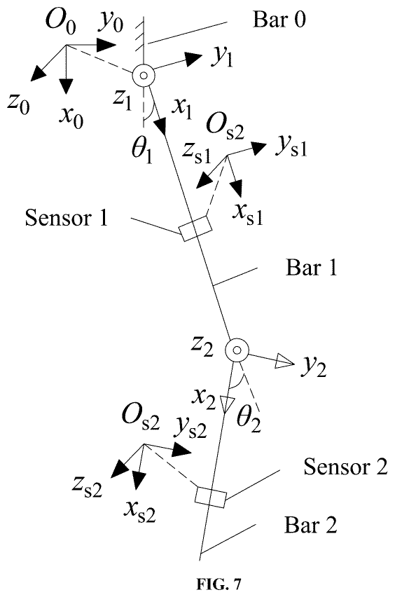

As shown in FIG. 7, taking the right leg as an example, the hip and knee joints are simplified into revolutes. Based on the D-H method, a fixed coordinate system O₀x₀y₀z₀, whose posture is the same as that of a navigation coordinate system of the wearable module, is established at the rotation center of the hip joint, where the x₀ axis points vertically downward from the rotation center O₀ of the hip joint toward the ground, the y₀ axis is perpendicular to the x₀ axis, pointing forward from O₀ toward the human body, and the z₀ axis is determined by the right-hand rule. Coordinate systems Oᵢxᵢyᵢzᵢ (i=1,2) are established at the hip and knee joints respectively, where the xᵢ axis points from the bar i to the bar i+1, the zᵢ axis is in the same direction as the z₀ axis, and the yᵢ axis is determined by the right-hand rule. Sensor carrier coordinate systems Oₛⱼxₛⱼyₛⱼzₛⱼ (j=1,2) are established at positions where the wearable modules are placed. As shown in FIG. 7, the directions of the axes of the carrier coordinate systems Oₛⱼxₛⱼyₛⱼzₛⱼ (j=1,2) are the same as those of Oᵢxᵢyᵢzᵢ (i=1,2), there is no rotation transformation, so the carrier coordinate systems can represent posture information of the hip and knee joint, with joint variables of the bars denoted as θₖ(k=1,2).

Since there is no transformation between the sensor carrier coordinate systems and the navigation coordinate system, the movement angle of the hip joint is θ₁.

$${θ}_{1} = {α}_{S 1} .$$

α_{S1} is the variation value of the pitch angle of the sensor 1 (implantable intelligent hip joint prosthesis 202).

The angle θ₂ of the knee joint is directly obtained by the rotation matrix:

$${θ}_{2} = {α}_{S 2} - {θ}_{1} .$$

α_{S2} is the variation value of the pitch angle of the sensor 2 (the wearable module 101 outside the total knee joint prosthesis). Considering the positive/negative orientation of the sensor 1 and the sensor 2 during data acquisition, where flexion is positive and extension is negative, the angle θ₂ of the knee joint is ultimately calculated as:

$${θ}_{2} = {θ}_{1} - {α}_{s 2} = {α}_{s 1} - {α}_{s 2} .$$

By following the above steps, the angles of the hip and knee joints of the unaffected and affected legs are calculated. By comparing joint angle data of the unaffected and affected legs during the same gait cycle, early warning of compensatory movements in the unaffected leg can be given in time. Both the affected and unaffected lower limbs of the patient are highly correlated in joint angles compared with a healthy subject. Although the correlation coefficients of the joint angles in both the affected and unaffected limbs are high, their root mean square values are significantly large. This indicates pronounced gait differences between the patient and the healthy subject during walking.

Moreover, the joint angle cycle diagram is used for quantitative analysis. The joint angle cycle diagram can visually illustrate the movement relationships between different joints during gait. By analyzing the shape, area and closure properties of the cycle diagram, differences in the range of motion between the unaffected and affected legs can be obtained through the joint angle cycle diagram while assessing the stability and coordination of gait.

By arranging four in-vitro sensors on both thighs and calves in combination with the implantable joint prostheses, raw data of joint movement angles are acquired synchronously. The implantable modules and the in-vitro wearable modules work cooperatively to achieve real-time synchronization of in-vivo and in-vitro data. The device is trained by the random forest machine learning algorithm based on the movement data of the healthy subject and the patient, so that the device can accurately recognize normal and abnormal movement patterns. Moreover, the system integrates the joint data of the implantable modules with kinematic data of the in-vitro wearable modules on the healthy leg and upper limb by artificial intelligence (AI) algorithms for comprehensive analysis.

The clinician can obtain the movement data of the patient in real time through a cloud platform and combine the movement data with clinical information to develop a precise rehabilitation plan and lifestyle adjustment strategy for the patient. This lower limb joint health assessment based on data analysis can effectively prevent the patient from relying too much on the healthy leg for compensatory activities, thereby reducing the risk of wear and tear of artificial joints.

The above steps are described in detail below.

Step 1: A power switch of the in-vitro multi-node wearable module is turned on such that the in-vitro multi-node wearable module starts to work.

Step 2: The sensors are set to a normal power mode through a program, and power modes of the accelerometer, the gyroscope and the magnetometer in the first IMU chip are set.

Step 3: The accelerometer (acceleration range, bandwidth, etc.) is configured. The magnetometer (data output rate, operating mode, etc.) is configured. The gyroscope (gyro range, bandwidth, etc.) is configured. Offset values of the first IMU chip (sOffsetAcc, sOffsetMag and sOffsetGyr) are set. The first IMU chip is calibrated.

Step 4: The first IMU chip acquires in-vitro data such as triaxial acceleration and triaxial gyroscope data, and transmits the in-vitro data to the first main control chip based on I2C.

Step 5: The first main control chip preprocesses the received raw data, where the preprocessing includes filtering and denoising.

Step 6: After the data are preprocessed, a pitch angle and a roll angle are calculated based on the acceleration data using Euler's law of motion. The angular velocity provided by the gyroscope is integrated to obtain the angle. The magnetometer may be used to correct a yaw angle.

Step 7: The pitch, roll and yaw data are transmitted to the Bluetooth chip through serial communication. The Bluetooth chip then wirelessly broadcasts the data to the mobile terminal.

Step 8: The lithium battery of the wearable module outside the joint prosthesis is connected to a direct current to alternating current converter, such that direct current power is converted into alternating current power. The electrical energy is then amplified by the power amplifier. The amplified electrical energy is radiated by the power supply inductive coil Q2 into the in-vivo implantable intelligent joint prosthesis by electromagnetic coupling.

Step 9: After receiving the alternating current power, the power supply inductive coil Q2 in the wearable module outside the joint prosthesis converts the alternating current power to direct current power through a rectification module of the NFC tag chip. A voltage regulator module then supplies the stable direct current power to all components of the in-vivo implantable intelligent joint prosthesis.

Step 10: The second IMU chip in the in-vivo implantable intelligent joint prosthesis transmits sensing data to the second main control chip by multiple communication modes.

Step 11: After filtering and denoising the data, the second main control chip encrypts the data. The encrypted data are packetized and transmitted in blocks to the NFC tag chip.

Step 12: The NFC tag chip stores the data cyclically in the EEPROM and overwrites the data cyclically after the storage space is full.

Step 13: After the read inductive coil Q1 and a capacitor are matched to achieve a 13.56 MHz resonance frequency, information stored in the NFC read/write chip can be wirelessly read by frequency-shift keying (PSK) modulation.

Step 14: The read information stored in the NFC read/write chip is transmitted to the mobile terminal through the Bluetooth chip.

Step 15: Based on monitoring data from the in-vitro multi-node wearable module and the in-vivo implantable intelligent joint prosthesis, the clinician performs multidimensional comparisons between in-vivo and in-vitro data or data between different in-vitro body parts.

Step 16: Based on multidimensional comparison results, the clinician adjusts the rehabilitation plan in real time while considering gender, age group and individual physical variations, thereby providing an individualized treatment schedule for the patient.

Step 17: The individualized rehabilitation and treatment schedule is made based on postoperative recovery time and individual variations. Using data obtained by the IMU chip, methods such as peak detection and velocity integration are employed to calculate cadence, step length and step speed.

Step 18: Based on the monitored data and postoperative period, early-stage training includes isometric contraction exercises for thigh muscles such as quadriceps femoris, knee extension exercises (small-range extension), mild knee flexion exercises, hip abduction and adduction, and hip flexion/extension exercises. This prevents excessive hip flexion (e.g., exceeding 90 degrees) to avoid the risk of hip dislocation. During the rehabilitation process, whether rehabilitation exercises achieve the predetermined goals are monitored in real time based on sensor data.

From the first day to one week after the surgical operation, the knee joint of the patient can achieve 90 degrees of flexion, which can be directly monitored by the device.

Step 19: During the middle stage of rehabilitation, the patient starts low-intensity gait training, gradually expanding the range of gait while using a walking assistance device (e.g., a walking aid or a walking stick). The patient increases the range of knee flexion and extension to gradually improve the mobility of the knee, with the goal of restoring normal gait. The patient starts to practice standing and walking while using a walking aid or a walking stick to reduce the load. The patient gradually increases the step length and frequency to improve the physical capacity. The patient continues with gait training to facilitate recovery of normal walking patterns. The patient performs progressive balance and coordination exercises to improve the ability to perform daily activities.

Step 20: Long-term rehabilitation and maintenance: The patient continues strengthening thigh muscles, especially the quadriceps femoris and gluteal muscles, and continues strengthening muscles around the hip, especially hip abductors and hip flexors. The patient should maintain a healthy weight and avoid excessive load. The patient should continue low-impact exercises (such as swimming and cycling) to maintain joint function and flexibility.

Step 21: Throughout all rehabilitation stages, especially during long-term rehabilitation and maintenance, the in-vitro multi-node wearable module and the in-vivo implantable intelligent joint prosthesis constantly monitors the joint, and the clinician sets peak values for the motion angle of the joint, step length, cadence and gait based on individual variations to prevent overload.

Step 22: The system dynamically adjusts the sensor monitoring frequency and data processing algorithms in the prosthesis based on the feedback of the clinician and the usage of the patient. This adjustment mechanism can optimize the monitoring accuracy and efficiency of the sensors in different rehabilitation stages, thereby ensuring data acquisition and processing to be matched with the actual needs of the patient and providing more accurate health information.

Step 23: The system can wirelessly update software in the prosthesis periodically. This feature allows timely integration of new algorithms, functions and performance improvements, so that the prosthesis can constantly adapt to the latest medical research findings and technological advancements. This wireless update mechanism also eliminates the need for the patient to frequently return to the hospital for physical examinations or software upgrades, thereby improving the convenience and satisfaction of the patient.

It is to be noted that the information of a user (including but not limited to device information of the user, personal information of the user and the like) and data (including but not limited to data for analysis, data for storage, data for exhibition and the like) in this application are information and data authorized by the user or fully authorized by each party, and the information and data are acquired, used and processed according to relevant regulations.

Those of ordinary skill in the art may understand that all or some of the procedures in the method of the foregoing embodiments may be implemented by a computer program instructing related hardware. The computer program may be stored in a nonvolatile computer-readable storage medium. When the computer program is executed, the procedures in the embodiments of the foregoing method may be performed. Any reference to a memory, a database, or other media used in the embodiments of this application may include a non-volatile and/or volatile memory. The nonvolatile memory may include a read-only memory (ROM), a magnetic tape, a floppy disk, a flash memory, an optical memory, a high-density embedded nonvolatile memory, a resistive random access memory (ReRAM), a magnetoresistive random access memory (MRAM), a ferroelectric random access memory (FRAM), a phase change memory (PCM), a graphene memory, etc. The volatile memory may include a random access memory (RAM) or an external cache memory. As an illustration rather than a limitation, the RAM may be in various forms, such as a static random access memory (SRAM) or a dynamic random access memory (DRAM).

The database in the embodiments of this application may include at least one of a relational database and a non-relational database. The non-relational database may include a distributed database based on a blockchain, but is not limited thereto. The processor in the embodiments of this application may be a general processor, a central processor, a graphics processor, a digital signal processor (DSP), a programmable logic device, and a data processing logic device based on quantum computing, but is not limited thereto.

The technical characteristics of the above embodiments can be employed in arbitrary combinations. To provide a concise description of these embodiments, all possible combinations of all the technical characteristics of the above embodiments may not be described; however, these combinations of the technical characteristics should be construed as falling within the scope defined by the specification as long as no contradiction occurs.

Several examples are used herein for illustration of the principles and implementations of this application. The description of the foregoing examples is used to help illustrate the method of this application and the core principles thereof. In addition, those of ordinary skill in the art can make various modifications in terms of specific implementations and scope of application in accordance with the teachings of this application. In conclusion, the content of the present specification shall not be construed as a limitation to this application.

What is claimed is:

1. An intelligent joint monitoring device integrating an in-vitro multi-node wearable module and an in-vivo implantable intelligent joint prosthesis, comprising an in-vitro multi-node wearable module and an in-vivo implantable intelligent joint prosthesis; wherein the in-vitro multi-node wearable module is configured to acquire movement data of a plurality of different in-vitro parts to form overall body posture and movement status data; the in-vivo implantable intelligent joint prosthesis is configured to acquire interarticular relative local movement status data; the in-vitro multi-node wearable module and the in-vivo implantable intelligent joint prosthesis cooperatively analyze a rehabilitation status of a patient with an implanted artificial joint;
the in-vitro multi-node wearable module comprises a first main control chip, a first inertial measurement unit (IMU) chip and a Bluetooth chip; the first IMU chip is configured to acquire in-vitro data, the first main control chip is connected to the first IMU chip, the first main control chip is configured to preprocess the in-vitro data, the Bluetooth chip is connected to the first main control chip, and the Bluetooth chip is configured to wirelessly broadcast the preprocessed in-vitro data to a mobile terminal; and
the in-vivo implantable intelligent joint prosthesis comprises a joint prosthesis body, and a second main control chip, a second IMU chip and a near field communication (NFC) tag chip that are arranged in the joint prosthesis body; and the second IMU chip is configured to acquire joint movement data in real time, the second main control chip is connected to the second IMU chip, the second main control chip is configured to preprocess and encrypt the joint movement data, the NFC tag chip is connected to the second main control chip, and the NFC tag chip is configured to store the encrypted joint movement data.

2. The intelligent joint monitoring device integrating an in-vitro multi-node wearable module and an in-vivo implantable intelligent joint prosthesis according to claim 1, wherein the in-vitro multi-node wearable module further comprises a lithium battery, wherein the lithium battery is configured to supply power to the first main control chip, the first IMU chip and the Bluetooth chip.

3. The intelligent joint monitoring device integrating an in-vitro multi-node wearable module and an in-vivo implantable intelligent joint prosthesis according to claim 2, wherein the in-vitro multi-node wearable module comprises a wearable module outside the joint prosthesis and a wearable module for other body parts.

4. The intelligent joint monitoring device integrating an in-vitro multi-node wearable module and an in-vivo implantable intelligent joint prosthesis according to claim 3, wherein the wearable module outside the joint prosthesis comprises a wearable module outside a total knee joint prosthesis and a wearable module outside a hip joint prosthesis; and the wearable module for other body parts comprises a torso wearable module, a leg wearable module and an arm wearable module.

5. The intelligent joint monitoring device integrating an in-vitro multi-node wearable module and an in-vivo implantable intelligent joint prosthesis according to claim 3, wherein the wearable module outside the joint prosthesis further comprises an NFC read/write chip, a power amplifier, a power supply inductive coil and a read inductive coil; the lithium battery supplies power to the in-vivo implantable intelligent joint prosthesis through the power amplifier and the power supply inductive coil; and the read inductive coil is configured to read information stored in the NFC read/write chip.

6. The intelligent joint monitoring device integrating an in-vitro multi-node wearable module and an in-vivo implantable intelligent joint prosthesis according to claim 5, wherein an inductive coil is further arranged in the joint prosthesis body, and the inductive coil is configured to receive electrical energy transferred by the power supply inductive coil.

7. The intelligent joint monitoring device integrating an in-vitro multi-node wearable module and an in-vivo implantable intelligent joint prosthesis according to claim 1, wherein the in-vivo implantable intelligent joint prosthesis comprises an in-vivo implantable intelligent total knee joint prosthesis and an in-vivo implantable intelligent hip joint prosthesis; and the joint prosthesis body comprises a total knee joint prosthesis body and a hip joint prosthesis body.

8. The intelligent joint monitoring device integrating an in-vitro multi-node wearable module and an in-vivo implantable intelligent joint prosthesis according to claim 7, wherein a temperature sensor, an air pressure sensor, a pH sensor, an impedance sensor and a blood pressure and blood oxygen chip are further arranged in the total knee joint prosthesis body.

9. An intelligent joint monitoring method integrating an in-vitro multi-node wearable module and an in-vivo implantable intelligent joint prosthesis, comprising:
acquiring, by an in-vitro multi-node wearable module, in-vitro data;
preprocessing the in-vitro data;
wirelessly broadcasting preprocessed in-vitro data to a mobile terminal;
acquiring, by an in-vivo implantable intelligent joint prosthesis, joint movement data, wherein the joint movement data comprises knee joint movement data and hip joint movement data;
preprocessing the joint movement data, wherein the preprocessing comprises filtering and denoising;
encrypting preprocessed joint movement data; and
transmitting the encrypted joint movement data to the mobile terminal.

10. The intelligent joint monitoring method integrating an in-vitro multi-node wearable module and an in-vivo implantable intelligent joint prosthesis according to claim 9, further comprising:
decrypting the encrypted joint movement data; and
monitoring a rehabilitation status of a patient in real time according to the decrypted joint movement data and the preprocessed in-vitro data, and adjusting a rehabilitation plan of the patient, wherein the rehabilitation status comprises an upper limb rehabilitation status and a lower limb rehabilitation status.

11. The intelligent joint monitoring method integrating an in-vitro multi-node wearable module and an in-vivo implantable intelligent joint prosthesis according to claim 10, wherein a process of monitoring the lower limb rehabilitation status comprises:
establishing a coordinate system based on a Denavit-Hartenberg (D-H) method, building a 6-degree-of-freedom (DOF) linkage, and using the 6-DOF linkage as a lower limb kinematic model, wherein the 6-DOF linkage has three rotational degrees of freedom, and the linkage is used for characterizing flexion and extension movements of a hip joint and flexion and hyperextension movements of a knee joint;
calculating, based on preprocessed in-vitro data of lower limbs, hip and knee joint angles of unaffected and affected legs according to the lower limb kinematic model;
training, with gait recognition as an objective, a random forest machine learning algorithm by using the hip and knee joint angles of the unaffected and affected legs and a triaxial acceleration and a triaxial angular velocity in the joint movement data as inputs, and monitoring results of the lower limb rehabilitation status as outputs, wherein types of the joint movement data comprise posture angle, the triaxial acceleration, the triaxial angular velocity, temperature, pH and impedance; and
obtaining the hip and knee joint angles of the monitored lower limbs and the triaxial acceleration and the triaxial angular velocity in the joint movement data, and recognizing the lower limb rehabilitation status by using the trained random forest machine learning algorithm, to obtain the monitoring results of the monitored lower limb rehabilitation status.
